(19) United States
(12) Patent Application Publication
(10) Pub. No.: US 2022/0287946 A1
(43) Pub. Date: Sep. 15, 2022

(54) ANHYDROUS SKIN CARE COMPOSITION COMPRISING RETINOL AND/OR BAKUCHIOL

(71) Applicant: Peace Out Inc., San Francisco, CA (US)

(72) Inventors: Enrico Frezza, Stateline, NV (US); Bobbie Marshall, Novato, CA (US)

(21) Appl. No.: 17/692,686

(22) Filed: Mar. 11, 2022

Related U.S. Application Data
(60) Provisional application No. 63/159,588, filed on Mar. 11, 2021.

(51) Int. Cl. A61K 8/67; A61K 8/34; A61K 8/31; A61K 8/92; A61K 8/36; A61K 8/02; A61Q 19/00

(52) U.S. Cl. CPC A61K 8/671; A61K 8/347; A61K 8/31; A61K 8/922; A61K 8/361; A61K 8/0229; A61Q 19/00

(57) ABSTRACT

Anhydrous skin care compositions contain encapsulates, which in turn contain active agents, including retinol and/or bakuchiol. When formulated in a stick form and applied to the skin, the pressure applied to the stick during application deposits the skin care composition onto the skin. During application, some of the encapsulates contained in the composition collapse or rupture, releasing some of the retinol and/or bakuchiol payload onto the skin. As application continues, the encapsulates may break up into smaller particles that retain some of the payload of the active agent(s), which remain on the skin and deliver all or a portion of the retained payload to the skin over time, resulting in “extended release” of active agent(s) into the skin.

### CROSS-REFERENCE TO RELATED APPLICATION

This application claims priority to and the benefit of U.S. Patent Application No. 63/159,588, entitled “ANHYDROUS SKIN CARE COMPOSITION COMPRISING RETINOL AND/OR BAKUCHIOL,” which was filed on Mar. 11, 2021. The contents of the aforementioned application are incorporated herein in their entirety for all purposes.

### BACKGROUND

Retinol is a form of vitamin A that may be found in many over-the-counter skin care products, and is often considered as the gold standard for combating wrinkles and fighting acne. Retinol may break down into retinoic acid on the skin, which in turn may act as an antioxidant that helps reverse skin damage and premature signs of aging. Retinol has been shown to promote collagen production, encourage cell turnover, reduce wrinkles and dark spots, as well as fight acne. However, retinol may be irritating to the skin, causing side effects such as peeling, redness, dryness and sun sensitivity to the retinol application site. Such side effects may be more pronounced when retinol-containing products are applied to sensitive areas such as to the skin under the eyes, which can be as much as 10 times thinner than the skin on the rest of the face, making it more prone to the aforementioned side effects. Bakuchiol, which is an analog of retinol, has been found to be as effective as retinol at targeting fine lines, wrinkles and uneven skin tone, while being less irritating to the skin than retinol.

Known over-the-counter products containing retinol or bakuchiol are typically in the form of a cream, a lotion or a serum. Some creams and lotions comprise either oil-in-water or water-in-oil emulsions, which may require addition of heat during manufacture. Some serums comprise water with relatively high levels of silicones, which may provide for a silky feel, but may not deliver any particular skin care benefits. Formulating such creams, lotions or serums so that any retinol and/or bakuchiol contained therein can be challenging, since the stability of both retinol and bakuchiol is known to be negatively impacted by exposure to heat, air, light and water. Over the course of use, a consumer may unwittingly subject such a cream, lotion or serum to any one or more of these destabilizing factors through repeated opening and closing of a jar or bottle of such a product. By dipping the fingers or an application device into the product, a consumer may contaminate the cream or serum with microbes. Thus, maintaining efficacy of retinol and bakuchiol in creams or serums can be challenging to a formulator.

The application of a retinol-containing or bakuchiol-containing cream, lotion or serum to the skin, particularly to skin under the eye, may be characterized by several drawbacks. For example, it is important that such creams, lotions and serums be applied with special care, since they can be irritating to the eyes. Furthermore, once applied to the skin, such creams, lotions and serums are typically rubbed into the skin, which can promote undesirable tugging of the skin, particularly skin under the eye.

For these and other reasons, a need remains for skin care compositions that deliver skin care benefits of retinol and/or bakuchiol, while providing for stability of these actives so that they are effective from first consumer use to last consumer use. Such skin care compositions would desirably be in a form that is easy to apply to the skin as well as in a form that minimizes exposure of the retinol and/or bakuchiol to destabilizing elements. Desirably, such a skin care composition should be in a form that minimizes tugging to the skin, particularly tugging of skin in the under eye area, during application. Further, such a skin care composition that does not require application to the skin with fingers that can be contaminated with microbes would also be desirable. Moreover, such a skin care composition would not contain high levels of silicone.

While a variety of compositions and methods for formulating and applying retinol or bakuchiol to the skin have been made and used, it is believed that no one prior to the inventors has made or used an invention as described herein.

### BRIEF SUMMARY

In general, the present disclosure is directed to anhydrous skin care compositions comprising one or more encapsulates, which in turn contain retinol, bakuchiol and combinations thereof. The present disclosure is further directed to methods of using the anhydrous skin care compositions. Further, the present disclosure is directed to anhydrous skin care compositions in the form of a stick or balm.

Exemplary care compositions may comprise one or more encapsulates that are the same or different structurally and/or chemically, a carrier and a structuring agent, wherein the one or more encapsulates contain one or more active agents selected from: retinol, bakuchiol and combinations thereof, and the skin care composition is anhydrous. The carrier may coat the one or more encapsulates present in the skin care composition, deliver skin care benefits to the skin, provide for slow release of the one or more active agents and combinations thereof.

Exemplary methods of delivering skin care skin care benefits to the skin comprise applying any exemplary skin care compositions to the skin, particularly the under eye area of the skin.

### BRIEF DESCRIPTION OF THE DRAWINGS

It is believed the present invention will be better understood from the following description of certain examples taken in conjunction with the accompanying drawings:

FIG. 1 depicts a graphical representation of an exemplary skin care composition contained in a tube with a cap;

FIG. 2 depicts a graphical representation of a cross-section of the exemplary skin care composition in the tube with the cap of FIG. 1 along the line 2-2; and

FIG. 3 depicts a graphical representation of the exemplary skin care composition in the tube with the cap removed therefrom.

The drawings are not intended to be limiting in any way, and it is contemplated that various embodiments of the invention may be carried out in a variety of other ways, including those not necessarily depicted in the drawing. The accompanying drawing that is incorporated in and forms a part of the specification illustrates several aspects of the present invention, and together with the description serves to explain the principles of the invention.

### DETAILED DESCRIPTION

The following description of certain examples of the invention should not be used to limit the scope of the present invention. Other examples, features, aspects, embodiments and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

All percentages, parts and ratios as used herein, are by weight of the total composition, unless otherwise specified. All such weights, as they pertain to listed ingredients, are based on the active level and therefore, do not include solvents or by-products that may be included in commercially available materials, unless otherwise specified.

Numerical ranges as used herein are intended to include every number and subset of numbers within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9 and so forth.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made. The following description of certain examples of the invention should not be used to limit the scope of the present invention. Other examples, features, aspects, embodiments and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Unless otherwise noted herein, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and” and “the” include plural referents unless the context clearly dictates otherwise. Thus, for example, reference to “a method” includes a plurality of such methods.

As used herein, the terms “about” or “approximately” mean within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

“Comprising” as used herein, means that the various components, ingredients or steps, can be conjointly employed in practicing the present invention. Accordingly, the term “comprising” encompasses the more restrictive terms “consisting essentially of” and “consisting of.”

“Substantially free” as used herein, means no effective amount of a particular component or a particular class of components as is specified herein. “Anhydrous” as used herein means substantially free of water, or may comprise about 0.5 wt. % or less, from about 0.1 wt. % or less, or even from about 0.01 wt. % or less or 0% (i.e., completely free), of water.

The present skin care compositions provide for the skin care benefits that may be achieved through the application of actives selected from retinol, bakuchiol and combinations thereof to a consumer's skin, particularly to the skin under a consumer's eyes. Without wishing to be bound by theory, it has surprisingly been found that by encapsulating these active(s) and then formulating the encapsulates containing active(s) in an anhydrous composition in stick-form, exposure to destabilizing factors such as air, light and water are minimized if not eliminated, as is the need to touch the composition with the fingers, which may in turn deposit microbes into any unused skin care composition and/or onto the skin to which the composition is applied.

By virtue of being anhydrous, the present skin care compositions may also retard or eliminate the proliferation of microorganisms within the compositions, which can result in additional benefits over water-containing products. For example, since exemplary skin care compositions are anhydrous, proliferation of any microorganisms contained therein is suppressed due to the absence of water. As such, exemplary skin care compositions may not require addition of components (e.g., preservatives) like some water-containing compositions do. Moreover, since exemplary skin care compositions are anhydrous, they may not be required to be submitted to microbiological testing that would otherwise be required in the skin care industry for water-containing products.

Exemplary skin care compositions comprise the following: retinol and/or bakuchiol encapsulates, carrier, structuring agent and non-encapsulated skin care agent. Exemplary skin care compositions are anhydrous and in the form of a stick that is dispensed from a tube. These and other features of skin care compositions pursuant to the present disclosure are described hereinbelow.

Retinol and/or Bakuchiol Encapsulates

Exemplary skin care compositions comprise one or more encapsulated active agents selected from retinol, bakuchiol and combinations thereof (i.e., “retinol and/or bakuchiol encapsulates”). Without wishing to be bound by theory, it is believed that encapsulation of retinol and/or bakuchiol pursuant to the present disclosure, provides at least the following advantages. First, encapsulation provides protection of the retinol and/or bakuchiol from destabilizing external elements, e.g., air, light and/or water, as well as destabilization by other components present in the skin care composition. Second, encapsulation may provide for extended release of the active agent(s) contained therein. For example, when an exemplary skin care composition comprising retinol and/or bakuchiol encapsulates is applied to the skin in stick form as described hereinbelow, the pressure applied to the stick during application and/or the act of rubbing the stick against the skin deposits the skin care composition to the skin—these action(s) may initially cause encapsulates contained in the composition to collapse or rupture, initially releasing a first portion of the payload of the active(s) contained therein. As the application/rubbing continues, the encapsulates may break up into smaller particles that retain some of the payload of the active agent(s), which remain on the skin and may deliver all or a portion of the retained payload to the skin over time, resulting in “extended release” of active agent(s) into the skin. Third, by extending release of active agent(s), dosing of the skin may occur over time, instead of all at once, so that benefits of smoothing, wrinkle-fighting, firming, etc. may be obtained while reducing or eliminating irritating side effects. Exemplary compositions may comprise one or more different types of encapsulates, each of the encapsulates containing retinol, bakuchiol and combinations thereof.

Some exemplary encapsulates containing retinol, bakuchiol and combinations thereof, may contain further skin care agents (i.e., “encapsulated skin care agents”) including, but not limited to those that are otherwise described hereinbelow under the heading “Non-Encapsulated Skin Care Agents.” Accordingly, some exemplary skin care compositions may contain a skin care agent that is located within retinol and/or bakuchiol encapsulates as well as outside of those encapsulates, i.e., distributed within the carrier. Moreover, some exemplary skin care compositions may contain a skin care agent that is only located within retinol and/or bakuchiol encapsulates and not distributed within the carrier, or vice versa. Moreover, some exemplary skin care compositions may contain encapsulates of retinol, bakuchiol and combinations thereof, wherein the structure of the capsule that contains the retinol, bakuchiol and combinations thereof, is itself made of one or more skin care agents otherwise described herein below under the heading “Non-Encapsulated Skin Care Agents.” For example, some skin care compositions may contain encapsulates of retinol, bakuchiol and combinations thereof, wherein the structure of the capsule comprises any one or more of the components described hereunder and which are selected from: carrier; structuring agent; non-encapsulated skin care agents; and combinations thereof.

Further suitable encapsulate structures and compositions that can provide sufficient protection for retinol and/or bakuchiol and/or the extended release thereof may be used. Exemplary encapsulate structures are shown and described in U.S. Patent Publication Numbers: 2016/0128944 (to Vyome Therapeutics); 2008/0193393 (to Lipo Chemicals); and 2007/0248633 (to L'Oréal). Useful amounts of retinol and/or bakuchiol in an encapsulate may vary depending upon the type of encapsulate that is utilized in the present skin care compositions.

Any suitable amount of retinol and/or bakuchiol may be present in exemplary skin care compositions, so long as the amount provides for effective treatment of the skin and is not too irritating to the skin. Some exemplary skin care compositions may comprise active(s) selected from retinol, bakuchiol and combinations thereof at a total amount by weight percentage of the skin care compositions of from about 0.0001% to about 2.5%, from about 0.001% to about 2.00%, or from about 0.01% to about 1.75%.

A single type of encapsulate, or a mixture of different types of encapsulates, may be present in exemplary skin care compositions at a total amount by weight percentage of the skin care compositions of from about 0.005% to about 5.0%, from about 0.05% to about 4.0%, or from about 0.5% to about 3.0%. Exemplary encapsulates of use may be all the same or different in terms of structure and chemical composition, including the chemical composition of the encapsulate itself and/or the active(s) content of the encapsulates.

### Carrier

Exemplary skin care compositions comprise one more carriers that deliver retinol and/or bakuchiol encapsulates to the skin. Without wishing to be bound by theory, it is believed that when present in exemplary skin care compositions, carriers as described herein, coat the encapsulates present in the skin care compositions, thus adding an additional layer of protection that stabilizes the encapsulates. Advantageously, some if not all of the disclosed carriers may also deliver skin conditioning benefits when the skin care compositions are applied to the skin. For example, some exemplary carriers of use may serve as skin moisturizing agents that allow for penetration of the actives (e.g., retinol and/or bakuchiol) into the skin and in some exemplary skin care compositions, aid in slow release of the actives into the skin.

Exemplary carriers of use include emollients. Exemplary emollients of use may be selected from hydrocarbon emollients (including natural and/or synthetic hydrocarbons), triglycerides, esters and combinations thereof.

Exemplary hydrocarbon emollients of use may be selected from: paraffinic hydrocarbons, saturated hydrocarbons (alkanes); unsaturated hydrocarbons (alkenes, alkynes); and combinations thereof. Useful hydrocarbon emollients include, but are not limited to, those that are selected from: liquid paraffin; dodecane; isododecane; isohexadecane; isoeicosane; squalane; mineral oil; coconut alkanes; hydrogenated polydecene; mineral oil; sunflower oil; safflower oil; palm oil; coconut oil; castor oil; botanical oils; and combinations thereof.

Exemplary triglycerides of use may be selected from: caprylic/capric triglycerides; oleic acid; linoleic acid; linolenic acid; erucic acid; stearic acid; palmitic acid; capric acid; lauric acid; myristic acid; and combinations thereof.

Exemplary esters of use may be selected from: cetyl esters; jojoba esters; glycereth-8; olive oil decyl esters; diisostearyl malate; lauryl laurate; cetyl palmitate; behenyl behenate and combinations thereof.

Exemplary emollients of use may have any suitable melting point that provides for skin care composition having the desired melting temperature to provide for the desired payoff to the skin. For example, some exemplary emollients may have a melting temperature of about 65° C.

One or more exemplary carriers may be present in exemplary skin care compositions at a total amount by weight percentage of the skin care compositions of from about 45% to about 80%, from about 50% to about 75% or from about 60% to about 70%.

### Structuring Agent

Exemplary skin care compositions comprise one more structuring agents. Any one or more structuring agents may be of use, so long as they provide for a skin care composition having the desired melting temperature to provide for the desired payoff to the skin, for example a melting temperature of at about 65° C. Exemplary structuring agents may be selected from viscosity modifiers (including, but not limited to dry and/or wet binders), film-forming agents and combinations thereof. Exemplary structuring agents may be selected from natural sources, synthetic sources and combinations thereof.

Exemplary structuring agents of use may be selected from: high molecular weight hydrocarbons; waxes; long-chain alcohols containing 12-32 carbons; fatty acids; high molecular weight alkanes; polymers; particles; fiber network forming agents; and combinations thereof. Exemplary structuring agents may be derived from raw materials selected from: ozokerite; carnauba; candelilla; polyethylene; synthetic wax; plant based wax; hydrogenated oils; glycerides; lecithin; microcrystalline waxes; plant based waxes; and combinations thereof. Exemplary waxes of use may have a melting point of from about 0° C. to about 95° C., from about 40° C. to about 90° C. or from about 50° C. to about 75° C. Exemplary skin care compositions may comprise a total amount by weight percentage of the skin care compositions of from about 10% to about 40%, from about 15% to about 37% or from about 20% to about 33% of one or more structuring agents.

### Non-Encapsulated Skin Care Agent

In addition to containing retinol and/or bakuchiol encapsulates, exemplary compositions may comprise additional skin care agents that are not encapsulated and/or form a part of the structure of the encapsulate (i.e., “non-encapsulated skin care agents”). Additional skin care agents of use may be selected from: penetration enhancers or solvent; absorbents/mattifying agents; anti-inflammatory agents; bioactives; chelating agents; anti-microbial agents; antioxidants; occlusive agents; protectants; minerals; vitamins; botanical oils; and combinations thereof.

Exemplary skin care products may comprise one or more penetration enhancers or solvent agents. Without wishing to be bound by theory, it is believed that penetration enhancers or solvent agents promote the diffusion of skincare agents via transdermal delivery. Exemplary penetration enhancers or solvent agents of use in the present skin care compositions may be selected from glycols, alcohols, surfactants, fatty acids, sulfoxides, terpenes, pyrrolidones, ethers, 1,2-hexanediol, polysorbate 20, oleic acid, dimethyl isosorbide, ethanol, polyethylene glycol and combinations thereof.

Exemplary skin care products may comprise one or more absorbents/mattifying agents. Without wishing to be bound by theory, it is believed that absorbents/mattifying agents promote non-tacky feel, non-oily feel and/or matte feel and/or matte appearance to the skin. Exemplary absorbents/mattifying agents of use may be selected from: mica; silica; cellulose; silicates; starches; inorganic pigments; clays; acrylates; elastomers; polymers; and combinations thereof.

Exemplary skin care products may comprise one or more anti-inflammatory, bioactive, chelating and anti-microbial agents. Without wishing to be bound by theory, it is believed that these agents provide beneficial activity within the product itself and/or on the skin. Examples of such agents of use may be selected from: glycosaminoglycans; long-chain polymers of disaccharides; ceramides; peptides; amino acids; herbal extracts; sodium hyaluronate; soy; phenoxyethanol; parabens; EDTA (ethylenediaminetetraacetic acid); and combinations thereof.

Exemplary skin care products may comprise one or more antioxidants. Without wishing to be bound by theory, it is believed that antioxidants of use may serve a dual purpose of protecting actives present in exemplary skin care compositions and/or delivering skin care benefits to the skin. Exemplary antioxidants of use may be selected from: butylated hydroxy anisole (BHA); butylated hydroxytoluene (BHT); pentaerythrityl tetra-di-t-butyl hydroxyhydrocinnamate ubiquinone; astaxanthin; tocopherols; carotenoids; ferulic acid; tocopherol acetate; ascorbic acid; reservatol; retinoids; polyphenols; herbal constituents; and combinations thereof.

Exemplary skin care products may comprise one or more occlusive agents and/or protectants. Without wishing to be bound by theory, it is believed that occlusive agents and/or protectants provide a physical barrier to the epidermis of a consumer's skin, thus preventing undesired water loss therefrom. Occlusive and/or protectant agents of use may be selected from: sterols; polyhydric alcohols; phospholipids; glycerin; lecithin; Avena sativa extract; allantoin; zinc; silicones; and combinations thereof.

One or more of the aforementioned skin care agents may be present in exemplary skin care compositions at total amount by weight percentage of the skin care compositions of from about 0.001% to about 10%, from about 0.01% to about 8%, from about 0.1% to about 6%.

Exemplary skin care compositions may be substantially free of animal products and byproducts. Such exemplary skin care compositions may comprise carriers, structuring agents and/or skin care agents selected from raw materials that are: plant derived; synthetically derived; and combinations thereof.

### Product Forms & Methods of Use

Exemplary skin care compositions may be in any suitable form including, but not limited to in a form similar to a lip gloss or balm in a container such as a tin or cup, packaged with a dip applicator, or in the form of a stick, like a lipstick.

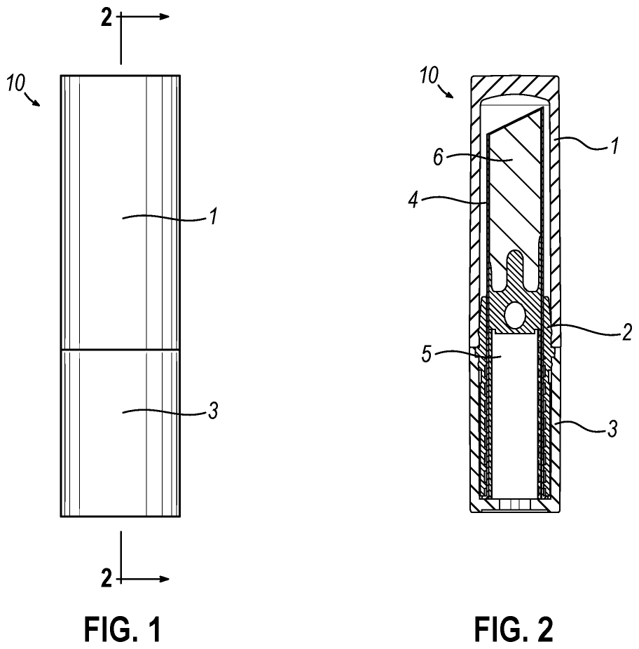

Referencing FIGS. 1-3, exemplary skin care compositions may be in the form of a stick that is stored in a tubular container 10. In the illustrated version, tubular container 10 comprises a lower tubular sleeve 5 and an upper tubular sleeve 4 positioned within a body 3. Lower tubular sleeve 5 is positioned within body 3 and is configured to receive the stick form of skin care composition 6 thereon. In the illustrated version, lower tubular sleeve 5 is translatable relative to body 3 to thereby translate composition 6, which in in the form of a tubular stick, within body 3. Upper tubular sleeve 4 is positioned within body 3 to extend from lower tubular sleeve 5 to outwardly of body 3 such that the stick form of composition 6 is configured to extend from lower tubular sleeve 5 through upper tubular sleeve 4. Upper tubular sleeve 4 can be coupled to body 3 via a coupling 2, such as an annular ring, to allow body 3 to rotate relative to upper tubular sleeve 4.

Accordingly, as body 3 is rotated relative to upper tubular sleeve 4, body 3 is configured to translate lower tubular sleeve 5 within body 3 such that lower tubular sleeve 5 can be raised and/or lowered relative to body 3 to thereby raise and/or lower composition 6 within body 3. In some versions, lower tubular sleeve 5 is threadably coupled with an interior surface of body 3 to allow lower tubular sleeve 5, and composition 6, to translate relative to body 3 when body 3 is rotated relative to upper tubular sleeve 4. Still other suitable configurations for translating composition 6 within container 10 will be apparent to one with ordinary skill in the art in view of the teachings herein. For instance, in some versions, lower tubular sleeve 5 can be pushed relative to body 3 to translate composition 6 within body 3 without rotating body 3 relative to upper tubular sleeve 4.

Tubular container 10 further comprises a cap 1 selectively couplable with body 3 such that cap 1 is configured to selectively cover composition 6. In the illustrated version, cap 1 is positioned on body 3 such that cap 1 is configured to receive upper tubular sleeve 4 within cap 1. Cap 1 can be couplable with body 3 by a friction fit or other suitable coupling (e.g., snap fit, threadable coupling, etc.) to allow cap 1 to be selectively coupled and/or removed with body 3.

To apply skin care composition 6, a user can remove cap 1 from body 3. The user can then expose a portion of the stick form of composition 6 from container 10. For instance, the user can grip upper tubular sleeve 4 and body 3 to rotate body 3 of container 10 in a first direction relative to upper tubular sleeve 4 to raise lower tubular sleeve 5 within body 3. Lower tubular sleeve 5 thereby raises composition 6 through upper tubular sleeve 4 to expose a portion of composition 6 above upper tubular sleeve 4. Once a portion of composition 6 is exposed, the exposed portion is applied by the user to the area of skin to be treated, e.g., the under eye area. The user may hold the stick on the lateral corner of the orbital socket near the hairline to warm the product and then glide medial towards the nose. The product is designed to be applied to the skin without the use of fingers, hands and/or a special applicator.

Once a desired quantity of an exemplary skin care composition is applied to the skin, the user may retract a remaining portion of the composition stick 6 back into container 10 by rotating body 3 of container 10 in a second direction relative to upper tubular sleeve 4, opposite to the first direction, to thereby retract lower tubular sleeve 5 within body 3. Lower tubular sleeve 5 thereby lowers composition 6 within body 3 such that composition 6 is positioned below upper tubular sleeve 4. Cap 1 can then be re-coupled with body 3 to cover composition 6. In the example shown in FIG. 1, cap 1 is replaced onto body 3 and pressed until a click is heard and/or felt by the user. The user may apply the product once a week up to once a day.

Dispensing exemplary skin care compositions from a tube as described herein may provide a number of benefits. For example, the stick may be stored inside the tube and only exposed to destabilizing elements, including but not limited to, air, light and water, during a brief product application period. Furthermore, during application of the skin care composition, the user need not use their fingers, hands and/or an applicator to apply the skin care composition to the skin, which may in turn minimize contamination of the skin care composition as well as the skin itself. In the exemplary skin care composition shown in FIG. 6, the opaque double barrel packaging may protect the composition from both air and light, while the dial applicator and click lock lid assure a tight seal when the packaging is closed.

### Method of Manufacture

Exemplary skin care compositions may be made as follows. Squalane, diisostearyl malate, waxes and other ingredients are added to a hopper with a side sweep and mixed for about 30 minutes at a temperature of from about 85° C. to about 90° C. Cosmetic actives are added to the hopper and mixing continues for about 5 to about 10 minutes at a temperature of from about 85° C. to about 90° C. Encapsulated retinol is added to the hopper and mixing continues for about 5 minutes at a temperature of from about 85° C. to about 90° C. Astaxanthin is added to the hopper and mixing continues for about 5 minutes at a temperature of from about 85° C. to about 90° C. The product is cooled and dispensed into a tubular container.

### Examples

Exemplary formulations for anhydrous skin care compositions comprising encapsulates that contain one or more active agents selected from: retinol, bakuchiol and combinations thereof are set forth in Table 2. Weight percentages that are set forth in Table 2 are by the total weight percentage of the resulting anhydrous skin care compositions.

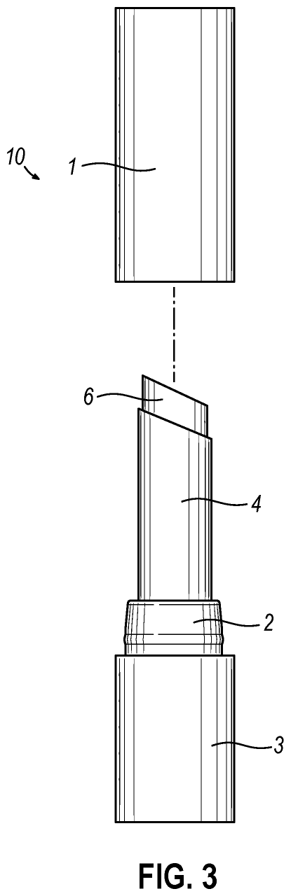

The resulting anhydrous skin care compositions exemplified in Table 1 are formed into any suitable shape including, for example, a tubular shape 6 as shown in FIGS. 2 and 3.

TABLE 1

| Component | | Ex. 1 | Ex. 2 | Ex. 3 | Ex. 4 | Ex. 5 | Ex. 6 | Ex. 7 | Ex. 8 |
|---|---|---|---|---|---|---|---|---|---|
| Category | Component | Wt % | Wt % | Wt % | Wt % | Wt % | Wt % | Wt % | Wt % |
| (A) Carrier | Squalane | 54 | 30 | 55 | 60 | 65 | 42 | 58 | 67 |
| | Caprylic/Capric | | 5 | | | | 10 | | 6 |
| | Triglyceride | | | | | | | | |
| | Diisostearyl Malate | 9 | 5 | 15 | 10 | 10 | | | 5 |
| | Coconut Alkanes | 2 | | 2 | | 2 | | 1.489 | 2 |
| | Botanical Oils | 15 | | | | | 15 | | |
| (B) Structuring | Synthetic Wax | 6 | 3 | 11.7 | 8 | 8 | | | 3 |
| Agent | Plant based waxes | 4 | 27 | 2 | 8 | 6 | 24 | 35 | 8 |
| | Lecithin | 1 | 2 | | 1.5 | 2 | 0.5 | 1 | 2 |
| | Hydrogenated Oils | | 7 | | | 5 | | 2 | 2 |
| | Brassica Glycerides | | 1 | 2 | | | 1 | 2 | 1.774 |
| (C) | Minerals + Vitamins | 2 | 3 | 5 | 3.95 | | 3 | | |
| Non-encapsulated | Antioxidant | 1.9995 | 0.5 | 0.1 | 0.05 | 0.001 | 0.5 | 0.001 | |
| Skin Care | Botanical Oils | 1 | 4 | 4 | 1.5 | | 1 | | |
| Agents | Ceramide | 0.4 | | | | | | | |
| | Peptides | 0.0005 | 0.01 | | 0.005 | 0.1 | | 0.005 | 0.001 |
| | Herbal Extracts | 1 | 0.79 | 0.4 | 2.5 | | | | |
| | Sodium Hyaluronate | 0.5 | 0.2 | | | | | | |
| | Bioactives | 1 | 0.5 | | | 0.998 | | | |
| | Anti-Microbial | 1.5 | 1 | 0.5 | 1.995 | 0.5 | 1 | 0.5 | |
| | Agents | | | | | | | | |
| (D) Encapsulates | Retinol | 0.1 | 0.5 | 0.05 | 0.0015 | 0.15 | 0.2 | 0.0001 | 0.3 |
| | Bakuchiol | 0.25 | 2 | 1 | 1.5 | | 0.5 | | 1 |
| | Cellulose Acetate | 0.2 | | | 0.4 | 0.2 | | 0.003 | 1.4 |
| | Butyrate | | | | | | | | |
| | Tricaprylin | 0.03 | | | 0.06 | | | 0.0001 | |
| | Pentaerythrityl | 0.02 | | | 0.04 | | 0.1 | 0.0018 | |
| | Tetra-di-t-butyl | | | | | | | | |
| | Hydroxyhydro- | | | | | | | | |
| | cinnamate | | | | | | | | |
| | Glycerin | | 0.75 | | | | 0.65 | | |
| | Caprylic/Capric | | 1 | | 0.44 | | | | 0.5 |
| | Triglyceride | | | | | | | | |
| | Polysorbate 20 | | 0.05 | | 0.05 | | | | |
| | Polycaprolactone | | 0.417 | | 0.0085 | | 0.4 | | |
| | Lecithin | | 0.1 | 0.5 | | | 0.05 | | |
| | Sodium Magnesium | | 0.178 | | | | 0.1 | | |
| | Silicate | | | | | | | | |
| | BHT | | 0.005 | | | 0.001 | | | 0.005 |
| | Phospholipids | | | 0.75 | | 0.05 | | | 0.02 |
| | Total Wt % | 100 | 100 | 100 | 100 | 100 | 100 | 100 | 100 |

A first exemplary skin care composition comprising:

a) one or more encapsulates that are the same or different structurally and/or chemically;
b) a carrier; and
c) a structuring agent;
wherein:
i. the one or more encapsulates contain one or more active agents selected from: retinol, bakuchiol and combinations thereof, and
ii. the skin care composition is anhydrous.

A second exemplary skin care composition according to the first exemplary skin care composition, comprising from about 0.005% to about 5.0% of encapsulates by total weight percentage of the skin care composition.

A third exemplary skin care composition according to either one of the first and second exemplary skin care compositions, comprising from about 0.0001% to about 2.5% of the one or more active agents by total weight percentage of the skin care composition.

A fourth exemplary skin care composition according to any one of the foregoing exemplary skin care compositions, wherein the skin care composition is in a form of a tubular stick.

A fifth exemplary skin care composition according to any one of the foregoing exemplary skin care compositions, wherein the carrier is selected from: hydrocarbon emollients; triglycerides; esters; and combinations thereof.

A sixth exemplary skin care composition according to the fifth exemplary skin care composition, wherein the carrier is a hydrocarbon emollient selected from: liquid paraffin; dodecane; isododecane; isohexadecane; isoeicosane; squalane; mineral oil; coconut alkanes; hydrogenated polydecene; mineral oil; sunflower oil; safflower oil; palm oil; coconut oil; castor oil; botanical oils; and combinations thereof.

A seventh exemplary skin care composition according to the fifth exemplary skin care composition, wherein the carrier is a triglyceride selected from: caprylic/capric triglycerides; oleic acid; linoleic acid; linolenic acid; erucic acid; stearic acid; palmitic acid; capric acid; lauric acid; myristic acid; and combinations thereof.

An eighth exemplary skin care composition according to any one of the foregoing exemplary skin care compositions, wherein the carrier is present in the skin care composition at a total amount by weight of the skin care composition of from about 45% to about 80%.

A ninth exemplary skin care composition according to any one of the foregoing exemplary skin care compositions, wherein the structuring agent is selected from: high molecular weight hydrocarbons; waxes; long-chain alcohols containing from 12 to 32 carbons; fatty acids; high molecular weight alkanes; polymers; particles; fiber network forming agents; and combinations thereof.

A tenth exemplary skin care composition according to according to any one of the foregoing exemplary skin care compositions, wherein the structuring agent is derived from raw materials selected from: ozokerite; carnauba; candelilla; polyethylene; synthetic wax; plant based wax; hydrogenated oils; glycerides; lecithin; microcrystalline waxes; plant based waxes; and combinations thereof.

An eleventh exemplary skin care composition according to according to any one of the foregoing exemplary skin care compositions, wherein the structuring agent is present in the skin care composition at a total amount by weight of the skin care composition of from about 10% to about 40%.

A twelfth exemplary skin care composition according to any one of the foregoing exemplary skin care compositions, further comprising non-encapsulated skin care agents selected from: penetration enhancers or solvent; absorbents/mattifying agents; anti-inflammatory agents; bioactives; chelating agents; anti-microbial agents; antioxidants; occlusive agents; protectants; minerals; vitamins; botanical oils; and combinations thereof.

A thirteenth exemplary skin care composition according to the twelfth exemplary skin care composition, wherein the non-encapsulated skin care agents are present in the skin care composition at a total amount by weight of the skin care composition of from about 0.001% to about 10%.

A fourteenth exemplary skin care composition, wherein the skin care composition is anhydrous and comprises:

a) one or more encapsulates that are the same or different structurally and/or chemically, wherein each of the one or more encapsulates contains skin care agents selected from: retinol; bakuchiol; and combinations thereof;
b) one or more carriers selected from: squalane; caprylic/capric triglyceride; diisostearyl malate; coconut alkanes; botanical oil; and combinations thereof; and
c) one or more structuring agents selected from: synthetic wax; plant based wax; lecithin; hydrogenated oils; brassica glycerides; and combinations thereof.

A fifteenth exemplary skin care composition according to the fourteenth exemplary skin care composition, comprising a total of from about 0.005% to about 5.0% of the encapsulates by total weight percentage of the skin care composition.

A sixteenth exemplary skin care composition according to either one of the fourteenth or exemplary skin care compositions, comprising from about 0.0001% to about 2.5% of the one or more active agents by total weight percentage of the skin care composition.

A seventeenth exemplary skin care composition, wherein the skin care composition is anhydrous and consists of:

a) a total of from about 0.5% to about 3.0% by weight percentage of the skin care composition of one or more encapsulates that are the same or different structurally and/or chemically, wherein each of the one or more encapsulates contains skin care agents selected from: retinol; bakuchiol; and combinations thereof;
b) a total of from about 60% to about 70% by weight percentage of the skin care composition of one or more carriers selected from: squalane; caprylic/capric triglyceride; diisostearyl malate; coconut alkanes; botanical oil; and combinations thereof;
c) a total of from about 20% to about 33% by weight percentage of the skin care composition of one or more structuring agents selected from: synthetic wax; plant based wax; lecithin; hydrogenated oils; brassica glycerides; and combinations thereof; and
d) a total of from about 0.1% to about 6% by weight percentage of the skin care composition of one or more non-encapsulated skin care agents selected from penetration enhancers or solvent; absorbents/mattifying agents; anti-inflammatory agents; bioactives; chelating agents; anti-microbial agents; antioxidants; occlusive agents; protectants; minerals; vitamins; and combinations thereof.

An eighteenth exemplary skin care composition, wherein the skin care composition is anhydrous and consists essentially of:

a) a total of from about 0.5% to about 3.0% by weight percentage of the skin care composition of one or more encapsulates that are the same or different structurally and/or chemically, wherein each of the one or more encapsulates contains skin care agents selected from: retinol; bakuchiol; and combinations thereof;
b) a total of from about 60% to about 70% by weight percentage of the skin care composition of one or more carriers selected from: squalane; caprylic/capric triglyceride; diisostearyl malate; coconut alkanes; botanical oil; and combinations thereof;
c) a total of from about 20% to about 33% by weight percentage of the skin care composition of one or more structuring agents selected from: synthetic wax; plant based wax; lecithin; hydrogenated oils; brassica glycerides; and combinations thereof and
d) a total of from about 0.1% to about 6% by weight percentage of the skin care composition of one or more non-encapsulated skin care agents selected from penetration enhancers or solvent; absorbents/mattifying agents; anti-inflammatory agents; bioactives; chelating agents; anti-microbial agents; antioxidants; occlusive agents; protectants; minerals; vitamins; and combinations thereof.

A nineteenth exemplary skin care composition according to any one of the foregoing exemplary skin care compositions, wherein the skin care composition is in a form of a stick.

A twentieth exemplary skin care composition according to the nineteenth exemplary skin care composition, wherein the stick is configured to be deployed from a tubular container.

A twenty-first exemplary skin care composition according to any one of the first through seventeenth exemplary skin care compositions, wherein the skin care composition is in a form of a balm.

A twenty-second exemplary skin care composition according any one of the foregoing exemplary skin care compositions, wherein the carrier:

(a) coats the one or more encapsulates present in the skin care composition;
(b) delivers skin care benefits to the skin;
(c) provides for slow release of the one or more active agents; or
(d) any combination of two or more of (a), (b) and (c).

A twenty-third exemplary skin care composition, according to any one of the foregoing exemplary skin care compositions, wherein the exemplary skin care compositions are substantially free of animal products and byproducts.

A first method of delivering skin care benefits to skin, the method comprising applying any one of the aforementioned exemplary skin care compositions to the skin.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those of ordinary skill in the art. For instance, the examples, embodiments, geometrics, materials, dimensions, ratios, steps, and the like discussed above, are illustrative and are not required. Accordingly, the scope of the present invention should be considered in terms of any claims that may be presented and is understood not to be limited to the details of structure and operation shown and described in the specification and drawings.

I claim/We claim:

1. A skin care composition comprising:
a. one or more encapsulates that are the same or different structurally and/or chemically;
b. b) a carrier; and
c. c) a structuring agent;
wherein:
i. i. the one or more encapsulates contain one or more active agents selected from: retinol, bakuchiol and combinations thereof, and
ii. the skin care composition is anhydrous.

2. The skin care composition of claim 1, comprising from about 0.005% to about 5.0% of encapsulates by total weight percentage of the skin care composition.

3. The skin care composition of claim 1, comprising from about 0.0001% to about 2.5% of the one or more active agents by total weight percentage of the skin care composition.

4. The skin care composition of claim 1, wherein the skin care composition is in a form of a tubular stick.

5. The skin care composition of claim 1, wherein the carrier is selected from: hydrocarbon emollients; triglycerides; esters; and combinations thereof.

6. The skin care composition of claim 5, wherein the carrier is a hydrocarbon emollient selected from: liquid paraffin; dodecane; isododecane; isohexadecane; isoeicosane; squalane; mineral oil; coconut alkanes; hydrogenated polydecene; mineral oil; sunflower oil; safflower oil; palm oil; coconut oil; castor oil; botanical oils; and combinations thereof.

7. The skin care composition of claim 5, wherein the carrier is a triglyceride selected from: caprylic/capric triglycerides; oleic acid; linoleic acid; linolenic acid; erucic acid; stearic acid; palmitic acid; capric acid; lauric acid; myristic acid; and combinations thereof.

8. The skin care composition of claim 1, wherein the carrier is present in the skin care composition at a total amount by weight of the skin care composition of from about 45% to about 80%.

9. The skin care composition of claim 1, wherein the structuring agent is selected from: high molecular weight hydrocarbons; waxes; long-chain alcohols containing from 12 to 32 carbons; fatty acids; high molecular weight alkanes; polymers; particles; fiber network forming agents; and combinations thereof.

10. The skin care composition of claim 1, wherein the structuring agent is derived from raw materials selected from: ozokerite; carnauba; candelilla; polyethylene; synthetic wax; plant based wax; hydrogenated oils; glycerides; lecithin; microcrystalline waxes; plant based waxes; and combinations thereof.

11. The skin care composition of claim 1, wherein the structuring agent is present in the skin care composition at a total amount by weight of the skin care composition of from about 10% to about 40%.

12. The skin care composition of claim 1, further comprising non-encapsulated skin care agents selected from: penetration enhancers or solvent; absorbents/mattifying agents; anti-inflammatory agents; bioactives; chelating agents; anti-microbial agents; antioxidants; occlusive agents; protectants; minerals; vitamins; botanical oils; and combinations thereof.

13. The skin care composition of claim 12, wherein the non-encapsulated skin care agents are present in the skin care composition at a total amount by weight of the skin care composition of from about 0.001% to about 10%.

14. A skin care product comprising the skin care composition of claim 4, wherein the tubular stick is configured to be deployed from a tubular container.

15. A skin care product comprising the skin care composition of claim 1, wherein the skin care composition is in a form of a balm.

16. An anhydrous skin care composition comprising:
a. one or more encapsulates that are the same or different structurally and/or chemically, wherein each of the one or more encapsulates contains skin care agents selected from: retinol; bakuchiol; and combinations thereof;
b. one or more carriers selected from: squalane; caprylic/capric triglyceride; diisostearyl malate; coconut alkanes; botanical oil; and combinations thereof; and
c. one or more structuring agents selected from: synthetic wax; plant based wax; lecithin; hydrogenated oils; brassica glycerides; and combinations thereof.

17. The skin care composition of claim 16, comprising a total of from about 0.005% to about 5.0% of the encapsulates by total weight percentage of the skin care composition.

18. The skin care composition of claim 16, comprising from about 0.0001% to about 2.5% of the one or more active agents by total weight percentage of the skin care composition.

19. A method of treating skin comprising applying the skin care composition of claim 16 to skin.

20. An anhydrous skin care composition consisting of:
a. a total of from about 0.5% to about 3.0% by weight percentage of the skin care composition of one or more encapsulates that are the same or different structurally and/or chemically, wherein each of the one or more encapsulates contains skin care agents selected from: retinol; bakuchiol; and combinations thereof;
b. a total of from about 60% to about 70% by weight percentage of the skin care composition of one or more carriers selected from: squalane; caprylic/capric triglyceride; diisostearyl malate; coconut alkanes; botanical oil; and combinations thereof;
c. a total of from about 20% to about 33% by weight percentage of the skin care composition of one or more structuring agents selected from: synthetic wax; plant based wax; lecithin; hydrogenated oils; brassica glycerides; and combinations thereof; and
d. a total of from about 0.1% to about 6% by weight percentage of the skin care composition of one or more non-encapsulated skin care agents selected from penetration enhancers or solvent; absorbents/mattifying agents; anti-inflammatory agents; bioactives; chelating agents; anti-microbial agents; antioxidants; occlusive agents; protectants; minerals; vitamins; and combinations thereof.
